SEQUENCE LISTING (8 sequences)

SEQ ID NO: 1 - DNA, 20 bp, synthetic construct
  source 1..20: mol_type = other DNA; note = P5 amplification primer
aatgatacggcgaccaccga

SEQ ID NO: 2 - DNA, 21 bp, synthetic construct
  source 1..21: mol_type = other DNA; note = P7 amplification primer
caagcagaagacggcatacga

SEQ ID NO: 3 - DNA, 20 bp, synthetic construct
  source 1..20: mol_type = other DNA; note = complementary P5 adapter
tcggtggtcgccgtatcatt

SEQ ID NO: 4 - DNA, 21 bp, synthetic construct
  source 1..21: mol_type = other DNA; note = complementary P7 adapter
tcgtatgccgtcttctgcttg

SEQ ID NO: 5 - DNA, 24 bp, synthetic construct
  source 1..24: mol_type = other DNA; note = PX primer
aggaggaggaggaggaggaggagg

SEQ ID NO: 6 - DNA, 30 bp, synthetic construct
  source 1..30: mol_type = other DNA; note = PY primer
gaagaagaagaagaagaagaagaagaagaa

SEQ ID NO: 7 - DNA, 24 bp, synthetic construct
  source 1..24: mol_type = other DNA; note = cPX adapter
cctcctcctcctcctcctcctcct

SEQ ID NO: 8 - DNA, 30 bp, synthetic construct
  source 1..30: mol_type = other DNA; note = cPY adapter
ttcttcttcttcttcttcttcttcttcttc

(19) United States
(12) Patent Application Publication
(10) Pub. No.: US 2025/0011834 A1
(43) Pub. Date: Jan. 9, 2025

(54) METHODS OF FUNCTIONALIZING MAGNETIC PARTICLES AND METHODS OF GENERATING AMPLICONS USING MAGNETIC PARTICLES

(71) Applicant: Illumina, Inc., San Diego, CA (US)

(72) Inventors: Teodora Gal, London (GB); Xavier von Hatten, Cambridge (GB); Gianluca Artioli, Cambridge (GB); Will Tovey, Bury St Edmunds (GB); Nam Nguyen, Cambridge (GB); Angelo La Rosa, Biggleswade (GB)

(73) Assignee: Illumina, Inc., San Diego, CA (US)

(21) Appl. No.: 18/753,740

(22) Filed: Jun. 25, 2024

Related U.S. Application Data
(60) Provisional application No. 63/511,129, filed on Jun. 29, 2023.

(51) Int. Cl. C12Q 1/6806; B01J 19/00; C12Q 1/6869; C12Q 1/6876

(52) U.S. Cl. CPC C12Q 1/6806; B01J 19/0046; C12Q 1/6869; C12Q 1/6876

(57) ABSTRACT

This application relates to functionalized magnetic particles and methods of using magnetic particles to generate amplicons. In some examples, a method of modifying a magnetic particle comprising a first functional group includes contacting the magnetic particle with a first molecule comprising a polymer coupled to second and third functional groups, wherein the first functional group reacts with the second functional group to form a bond via which the polymer is coupled to the magnetic particle. The method may include contacting the magnetic particle with a second molecule, the second molecule comprising an oligonucleotide coupled to a fourth functional group, wherein the third functional group reacts with the fourth functional group to form a bond via which the oligonucleotide is coupled to the magnetic particle.

### CROSS-REFERENCE TO RELATED APPLICATIONS

This application claims the benefit of U.S. Provisional Application No. 63/511,129, filed on Jun. 29, 2023 and entitled “Methods of Functionalizing Magnetic Particles and Methods of Generating Amplicons Using Magnetic Particles,” the entire contents of which are incorporated by reference herein.

### INCORPORATION BY REFERENCE OF SEQUENCE LISTING

The material in the accompanying sequence listing is hereby incorporated by reference into the application. The accompanying sequence listing XML file, named “IP-2554-US.xml”, was created on Jun. 2, 2024 and is 12 kB in size.

### FIELD

This application relates to methods of functionalizing particles and generating amplicons using particles.

### BACKGROUND

Cluster amplification is an approach to amplifying polynucleotides, for example for use in genetic sequencing. Target polynucleotides are captured by primers (e.g., P5 and P7 primers) coupled to a substrate surface in a flowcell, and form “seeds” at random locations on the surface. Cycles of amplification are performed to form clusters on the surface around each seed. The clusters include copies, and complementary copies, of the seed polynucleotides. In some circumstances, the substrate is patterned so as to define regions that bound different clusters, such as wells that may be filled with respective clusters.

### SUMMARY

Examples provided herein are related to functionalizing magnetic particles, and generating amplicons using magnetic particles.

Some examples herein provide a method of modifying a magnetic particle including a first functional group. The method may include contacting the magnetic particle with a first molecule including a polymer coupled to second and third functional groups. The first functional group reacts with the second functional group to form a bond via which the polymer is coupled to the magnetic particle. The method may include contacting the magnetic particle with a second molecule. The second molecule may include an oligonucleotide coupled to a fourth functional group. The third functional group reacts with the fourth functional group to form a bond via which the oligonucleotide is coupled to the magnetic particle.

In some examples, the oligonucleotide includes an amplification primer.

In some examples, the second and third functional groups are of the same type as one another. In some examples, the second and third functional groups are of different types than one another.

In some examples, the polymer includes a hydrogel. In some examples, the hydrogel includes polyacrylamide.

In some examples, the magnetic particle has a diameter of about 100 nm to about 300 nm.

Some examples herein provide a method of modifying a magnetic particle coupled to a polymer including a first functional group. The method includes contacting the magnetic particle with a molecule including an oligonucleotide coupled to a second functional group. The second functional group reacts with the first functional group to form a bond via which the oligonucleotide is coupled to the magnetic particle.

Some examples herein provide a composition made by any of the foregoing methods.

Some examples herein provide a composition that includes a magnetic particle, and a polymer coupled to the magnetic particle via a reaction product of a first functional group coupled to the magnetic particle and a second functional group coupled to the polymer. The composition also may include a plurality of oligonucleotides coupled to the polymer via reaction products of third functional groups coupled to the polymer and fourth functional groups coupled to the oligonucleotides.

Some examples herein provide a method of using such a composition. The method may include hybridizing a template polynucleotide to a first oligonucleotide of the plurality. The method may include amplifying the hybridized template polynucleotide using additional oligonucleotides of the plurality to generate a plurality of amplicons coupled to the polymer via reaction products of the third and fourth functional groups.

In some examples, the method may include, after the amplifying, disposing the magnetic particle within a flow cell. In some examples, disposing the magnetic particle within the flow cell includes electrostatically attracting the magnetic particle to a region of the flow cell.

In some examples, the method may include, before the amplifying, disposing the magnetic particle within a flow cell.

Some examples herein provide a device that include a flow cell; and any of the foregoing compositions located within the flow cell.

In some examples, the flow cell includes a positively charged surface to which the composition is noncovalently bound through an electrostatic force.

Some examples herein provide a composition that includes a magnetic particle; and a molecule coupled to the magnetic particle. The molecule may include a polymer coupled to the magnetic particle via a reaction product of first and second functional groups. The composition may include a plurality of polynucleotide amplicons coupled to the polymer via reaction products of third and fourth functional groups.

Some examples herein provide a device that includes a flow cell; and the foregoing composition located within the flow cell.

In some examples, the flow cell includes a positively charged surface to which the composition is noncovalently bound through an electrostatic force.

Some examples provide a method of using either of the foregoing devices. The method may include sequencing the plurality of polynucleotide amplicons within the flow cell.

Some examples herein provide a method of generating an amplicon. The method may include coupling to a first particle a first double-stranded polynucleotide including first and second strands hybridized to one another. The first strand may include a first amplification adaptor, and the second strand may include a second amplification adaptor that is complementary to the first amplification adapter. The method may include contacting the first particle with a second particle. The second particle may include a first amplification primer that is complementary to the first amplification adapter. The method may include using a recombinase to hybridize the first amplification primer to the first amplification adaptor of the first double-stranded polynucleotide. The method may include extending the first amplification primer to generate a first amplicon of the first strand of the first double-stranded polynucleotide.

In some examples, extending the first amplification primer releases the first strand from the first particle.

In some examples, the second particle further includes a second amplification primer that is complementary to the second amplification adaptor, the method further including using the second amplification primer to generate a second amplicon of the first strand of the first double-stranded polynucleotide. In some examples, the method further includes disposing the second particle within a flow cell. In some examples, disposing the second particle within the flow cell includes electrostatically attracting the second particle to a region of the flow cell. In some examples, the second amplicon is generated before the second particle is disposed within the flow cell. In some examples, the second amplicon is generated after the second particle is disposed within the flow cell. In some examples, the method further includes sequencing the first amplicon of the first strand of the first double-stranded polynucleotide within the flow cell.

In some examples, the first particle includes a carboxyl group to which the first double-stranded polynucleotide adsorbs.

In some examples, the first particle is magnetic, and the second particle is not magnetic. In some examples, the method further includes using a magnet to separate the first particle from the second particle.

In some examples, the first particle includes a solid phase reversible immobilization (SPRI) bead.

In some examples, the first particle is coupled to a second double-stranded polynucleotide including additional first and second strands hybridized to one another. The first strand may include the first amplification adaptor, and the additional second strand may include the second amplification adaptor. The method may include contacting the first particle with a third particle including the first amplification primer. The method may include using the recombinase to hybridize the first amplification primer of the third particle to the first amplification adaptor of the second double-stranded polynucleotide. The method may include extending the first amplification primer to generate a first amplicon of the first strand of the second double-stranded polynucleotide.

Some examples herein provide a composition. The composition may include a first particle coupled to a first double-stranded polynucleotide including first and second strands hybridized to one another. The first strand may include a first amplification adaptor. The second strand may include a second amplification adaptor that is complementary to the first amplification adapter. The composition may include a second particle in contact with the second particle and including a first amplification primer that is complementary to the first amplification adapter. The composition may include a recombinase to hybridize the first amplification primer to the first amplification adaptor of the first double-stranded polynucleotide.

Some examples herein provide a kit. The kit may include a first particle coupled to a first double-stranded polynucleotide including first and second strands hybridized to one another. The first strand may include a first amplification adaptor. The second strand may include a second amplification adaptor that is complementary to the first amplification adapter. The kit may include a second particle including a first amplification primer that is complementary to the first amplification adapter. The kit may include a recombinase to hybridize the first amplification primer to the first amplification adaptor of the first double-stranded polynucleotide.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

### BRIEF DESCRIPTION OF THE DRAWINGS

FIGS. 1A-1D schematically illustrate example structures and operations for preparing a magnetic particle coupled to a polymer, and optionally also to oligonucleotides.

FIG. 2 schematically illustrates an example of a workflow for generating amplicons using a magnetic particle.

FIGS. 3A-3H schematically illustrate an alternative example workflow for generating amplicons using a magnetic particle.

FIG. 4 schematically illustrates an alternative example workflow for capturing a

FIG. 5 provides data showing sequencing metrics (pass filter (% PF), %>Q30, and occupancy percentages) for particles that were clustered on a flowcell in accordance with FIG. 2.

FIG. 6A is a scanning electron microscopy (SEM) image of 250 nm coated and grafted magnetic particles after a sequencing run.

FIG. 6B is a SEM image of a 250 nm coated and grafted magnetic particle after a sequencing run.

FIGS. 7A-7B are SEM images of SPRI beads having PAZAM-coated and grafted particles coupled thereto via hybridization using recombinase.

FIG. 8A is an image of a sequencing lane generated using the workflow described with reference to FIG. 4, followed by on-board clustering using a recombinase described with reference to FIG. 2.

FIG. 8B is an image of a sequencing lane generated using the workflow described with reference to FIG. 8A, but without using recombinase, as a negative control.

FIG. 9 provides data showing primary sequencing metrics between OBC particles which were seeded in suspension in accordance with FIG. 2, and OBC particles which were seeded in accordance with FIG. 4.

### DETAILED DESCRIPTION

Examples provided herein are related to methods of functionalizing magnetic particles and generating amplicons using magnetic nanoparticles.

Cluster generation typically takes place on a sequencing instrument. However, when cluster generation is being performed, the sequencing instrument cannot be used to perform sequencing cycles. As a result, the instrument's data generation throughput is reduced. Methods that perform cluster generation in suspension and/or on particles before capturing on the sequencing instrument have the potential to increase sequencing throughput as well as enable alternative methods for library preparation.

Disclosed herein are methods of functionalizing magnetic particles that can be used to perform clustering in suspension, rather than on a sequencing instrument, thus facilitating throughput of the sequencing instrument. Also disclosed herein are methods of generating amplicons using magnetic particles.

Some examples herein relate to magnetic particles and their methods of preparation and use. Such magnetic particles optionally may be used to capture and amplify a polynucleotide in solution, and the particles (with amplicons of the polynucleotide attached) subsequently may be disposed on a flowcell for use in sequencing the amplicons, e.g., using sequencing-by-synthesis. Because the particles capture selectively within the nanowells, the construction of the flowcell may be significantly simplified relative to that of a flowcell that is coated and subsequently polished to remove the material sticking at the interstitials between the nanowells; for example, a flowcell for use with the present magnetic particles need not necessarily include depression patterned regions of capture primers, as may be the case for flowcells fabricated with previously known technology. After the magnetic particle captures the polynucleotide, amplification is performed at the surface of the magnetic particle where all the strands of a cluster are confined, and the particle may be disposed at any suitable location on the flowcell. Additionally, monoclonality of the clusters may be improved by performing seeding and clustering using magnetic particles in solution, for example because the magnetic particles may be expected to be sufficiently spaced apart from one another in the solution that a cluster of amplicons coupled to a given particle may not be expected to be able to be contaminated by amplicons coupled to another such particle; in comparison, performing seeding and clustering on a flowcell surface runs the risk that an amplicon from one cluster may contaminate another cluster, resulting in generation of a polyclonal cluster which may not be usable for sequencing. Additionally, the present magnetic particles may increase the speed with which sequencing may be performed, for example because the capture and amplification operations may be performed off-instrument without the need to utilize time on the instrument to perform such operations; in comparison, performing capture and amplification on the flowcell can utilize significant instrument time that otherwise could have been used for sequencing.

Magnetic particles are very easy to handle as magnetic pull may be used to manipulate them. In particular, by performing the polynucleotide capture and amplification separately, any polynucleotide strands which are not captured are washed away before amplification is performed. In some examples, this washing is performed by magnetically retaining the particles which have been used to capture polynucleotide strands, and washing away any solution components (e.g., non-captured polynucleotide strands) which are not coupled to the particles. In other examples, this washing is performed by using non-magnetic particles to capture polynucleotides off of magnetic particles, and magnetically retaining the residual magnetic particles and collecting the non-magnetic particles, which have captured respective polynucleotides, from solution. These and other examples provided herein may be used to inhibit any free-floating polynucleotides from being inadvertently amplified in solution and then being captured by particles, which otherwise may increase polyclonality.

First, some terms used herein will be briefly explained. Then, some methods for functionalizing magnetic particles and generating amplicons using magnetic particles, will be described.

### Terms

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

The terms “substantially,” “approximately,” and “about” used throughout this specification are used to describe and account for small fluctuations, such as due to variations in processing. For example, they may refer to less than or equal to ±10%, such as less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

As used herein, “hybridize” is intended to mean noncovalently associating a first polynucleotide to a second polynucleotide along the lengths of those polymers to form a double-stranded “duplex.” For instance, two DNA polynucleotide strands may associate through complementary base pairing. The strength of the association between the first and second polynucleotides increases with the complementarity between the sequences of nucleotides within those polynucleotides. The strength of hybridization between polynucleotides may be characterized by a temperature of melting (Tm) at which 50% of the duplexes have polynucleotide strands that disassociate from one another. Polynucleotides that are “partially” hybridized to one another means that they have sequences that are complementary to one another, but such sequences are hybridized with one another along only a portion of their lengths to form a partial duplex. Polynucleotides with an “inability” to hybridize include those which are physically separated from one another such that an insufficient number of their bases may contact one another in a manner so as to hybridize with one another.

As used herein, the term “nucleotide” is intended to mean a molecule that includes a sugar and at least one phosphate group, and in some examples also includes a nucleobase. A nucleotide that lacks a nucleobase may be referred to as “abasic.” Nucleotides include deoxyribonucleotides, modified deoxyribonucleotides, ribonucleotides, modified ribonucleotides, peptide nucleotides, modified peptide nucleotides, modified phosphate sugar backbone nucleotides, and mixtures thereof. Examples of nucleotides include adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxythymidine monophosphate (dTMP), deoxythymidine diphosphate (dTDP), deoxythymidine triphosphate (dTTP), deoxycytidine diphosphate (dCDP), deoxycytidine triphosphate (dCTP), deoxyguanosine monophosphate (dGMP), deoxyguanosine diphosphate (dGDP), deoxyguanosine triphosphate (dGTP), deoxyuridine monophosphate (dUMP), deoxyuridine diphosphate (dUDP), and deoxyuridine triphosphate (dUTP).

As used herein, the term “nucleotide” also is intended to encompass any nucleotide analogue which is a type of nucleotide that includes a modified nucleobase, sugar and/or phosphate moiety compared to naturally occurring nucleotides. Example modified nucleobases include inosine, xanthine, hypoxanthine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. As is known in the art, certain nucleotide analogues cannot become incorporated into a polynucleotide, for example, nucleotide analogues such as adenosine 5′-phosphosulfate. Nucleotides may include any suitable number of phosphates, e.g., three, four, five, six, or more than six phosphates.

As used herein, the term “polynucleotide” refers to a molecule that includes a sequence of nucleotides that are bonded to one another. A polynucleotide is one nonlimiting example of a polymer. Examples of polynucleotides include deoxyribonucleic acid (DNA), ribonucleic acid (RNA), and analogues thereof. A polynucleotide may be a single stranded sequence of nucleotides, such as RNA or single stranded DNA, a double stranded sequence of nucleotides, such as double stranded DNA, or may include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides may include non-naturally occurring DNA, such as enantiomeric DNA. The precise sequence of nucleotides in a polynucleotide may be known or unknown. The following are examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide, branched polynucleotide, plasmid, vector, isolated DNA of any sequence, isolated RNA of any sequence, nucleic acid probe, primer or amplified copy of any of the foregoing.

As used herein, a “polymerase” is intended to mean an enzyme having an active site that assembles polynucleotides by polymerizing nucleotides into polynucleotides. A polymerase can bind a primed single stranded target polynucleotide, and can sequentially add nucleotides to the growing primer to form a “complementary copy” polynucleotide having a sequence that is complementary to that of the target polynucleotide. Another polymerase, or the same polymerase, then can form a copy of the target nucleotide by forming a complementary copy of that complementary copy polynucleotide. Any of such copies may be referred to herein as “amplicons.” DNA polymerases may bind to the target polynucleotide and then move down the target polynucleotide sequentially adding nucleotides to the free hydroxyl group at the 3′ end of a growing polynucleotide strand (growing amplicon). DNA polymerases may synthesize complementary DNA molecules from DNA templates and RNA polymerases may synthesize RNA molecules from DNA templates (transcription). Polymerases may use a short RNA or DNA strand (primer), to begin strand growth. Some polymerases may displace the strand upstream of the site where they are adding bases to a chain. Such polymerases may be said to be strand displacing, meaning they have an activity that removes a complementary strand from a template strand being read by the polymerase. Exemplary polymerases having strand displacing activity include, without limitation, the large fragment of Bst (Bacillus stearothermophilus) polymerase, exo-Klenow polymerase or sequencing grade T7 exo-polymerase. Some polymerases degrade the strand in front of them, effectively replacing it with the growing chain behind (5′ exonuclease activity). Some polymerases have an activity that degrades the strand behind them (3′ exonuclease activity). Some useful polymerases have been modified, either by mutation or otherwise, to reduce or eliminate 3′ and/or 5′ exonuclease activity.

As used herein, the term “primer” is defined as a polynucleotide to which nucleotides may be added via a free 3′ OH group. A primer may include a 3′ block preventing polymerization until the block is removed. A primer may include a modification at the 5′ terminus to allow a coupling reaction or to couple the primer to another moiety. A primer may include one or more moieties which may be cleaved under suitable conditions, such as UV light, chemistry, enzyme, or the like. The primer length may be any suitable number of bases long and may include any suitable combination of natural and non-natural nucleotides. A target polynucleotide may include an “adapter” that hybridizes to (has a sequence that is complementary to) a primer, and may be amplified so as to generate a complementary copy polynucleotide by adding nucleotides to the free 3′ OH group of the primer.

A “capture primer” is intended to mean a primer that is coupled to a substrate and may hybridize to an adapter of the target polynucleotide. In some cases, a capture primer that is coupled to the substrate and may hybridize to another adapter of that target polynucleotide may be referred to as an “orthogonal capture primer.” The adapters may have respective sequences that are complementary to those of capture primers to which they may hybridize. A capture primer and an orthogonal capture primer may have different and independent sequences than one another. A capture primer that may be used to hybridize to an adapter of a target polynucleotide in order to couple that polynucleotide to the substrate, but that may not be used to grow a complementary strand during an amplification process, may in some cases be referred to as a “seeding primer.” A capture primer that may be used to grow a complementary strand during an amplification process may in some cases be referred to as an “amplification primer.”

As used herein, the term “substrate” refers to a material used as a support for compositions described herein. Example substrate materials may include glass, silica, plastic, quartz, metal, metal oxide, organo-silicate (e.g., polyhedral organic silsesquioxanes (POSS)), polyacrylates, tantalum oxide, complementary metal oxide semiconductor (CMOS), or combinations thereof. An example of POSS can be that described in Kehagias et al., Microelectronic Engineering 86 (2009), pp. 776-778, which is incorporated by reference in its entirety. In some examples, substrates used in the present application include silica-based substrates, such as glass, fused silica, or other silica-containing material. As used herein, by “silica” it is meant silicon dioxide with chemical composition of SiOx, where x is approximately equal to 2. Silica may be porous. In some examples, substrates may include silicon, silicon nitride, or silicone hydride. In some examples, substrates used in the present application include plastic materials or components such as polyethylene, polystyrene, poly(vinyl chloride), polypropylene, nylons, polyesters, polycarbonates, and poly(methyl methacrylate). Example plastics materials include poly(methyl methacrylate), polystyrene, and cyclic olefin polymer substrates. In some examples, the substrate is or includes a silica-based material or plastic material or a combination thereof. In particular examples, the substrate has at least one surface comprising glass or a silicon-based polymer. In some examples, the substrates may include a metal. In some such examples, the metal is gold. In some examples, the substrate has at least one surface comprising a metal oxide. In one example, the surface comprises a tantalum oxide or tin oxide. Acrylamides, enones, or acrylates may also be utilized as a substrate material or component. Other substrate materials may include, but are not limited to gallium arsenide, indium phosphide, aluminum, ceramics, polyimide, quartz, resins, polymers and copolymers. In some examples, the substrate and/or the substrate surface may be, or include, quartz. In some other examples, the substrate and/or the substrate surface may be, or include, semiconductor, such as GaAs or ITO. The foregoing lists are intended to be illustrative of, but not limiting to the present application. Substrates may comprise a single material or a plurality of different materials. Substrates may be composites or laminates. In some examples, the substrate comprises an organo-silicate material. Substrates may be flat, round, spherical, rod-shaped, or any other suitable shape. Substrates may be rigid or flexible.

In some examples, a substrate is or includes a particle. As used herein, a “particle” is a small localized object which exists as a discrete unit in a given medium. In detail, the term refers to microscopic particles with sizes ranging from atoms to molecules, such as nanoparticles or colloidal particles. A particle may refer to a substrate that is sufficiently small that it may be located within a fluid (such as a liquid), that is, substantially surrounded on all sides by the fluid. As such, a particle may be carried by (moved by) the fluid in which it is located, for example by flowing the fluid from one location to another location. As explained elsewhere herein, particles can have a variety of shapes. Optionally, particles may be porous. When a particle is at least partially spherical, it may be referred to as a “bead.” In some examples, a first substrate (such as a flat or patterned surface within a flowcell) may be used to support a second substrate (such as a particle).

In some examples, a substrate includes a patterned surface. A “patterned surface” refers to an arrangement of different regions in or on an exposed layer of a substrate. For example, one or more of the regions may include feature(s) that can be used to attract and/or be coupled to a particle. The feature(s) can be separated by interstitial regions where such features are not present. In some examples, the pattern may be an x-y format of features that are in rows and columns. In some examples, the pattern may be a repeating arrangement of features and/or interstitial regions. In some examples, the pattern may be a random arrangement of features and/or interstitial regions. In some examples, substrate includes an array of wells (depressions) in a surface, into which particles respectively may be disposed. The wells may be provided by substantially vertical sidewalls. Wells may be fabricated as is generally known in the art using a variety of techniques, including, but not limited to, photolithography, stamping techniques, molding techniques and microetching techniques. As will be appreciated by those in the art, the technique used will depend on the composition and shape of the array substrate.

The features in a patterned surface of a substrate may include wells in an array of features (e.g., microwells or nanowells) on glass, silicon, plastic or other suitable material(s) with a patterned, covalently-linked hydrogel such as poly(N-(5-azidoacetamidylpentyl) acrylamide-co-acrylamide) (PAZAM, which is a type of polyacrylamide). The process creates hydrogel regions used to attract particles for sequencing that may be stable over sequencing runs with a large number of cycles. In some examples, covalent linking of the hydrogel to the wells may be helpful for maintaining the hydrogel in the structured features throughout the lifetime of the structured substrate during a variety of uses. However in many examples, the hydrogel need not be fully or even partially covalently linked to the wells. For example, in some conditions silane free acrylamide (SFA, which is another type of polyacrylamide) may be used as the hydrogel material.

In particular examples, a structured substrate may be made by patterning a suitable material with wells (e.g. microwells or nanowells), coating the patterned material with a hydrogel material (e.g., PAZAM, SFA or chemically modified variant thereof, such as the azidolyzed version of SFA (azido-SFA)) and polishing the surface of the hydrogel coated material, for example via chemical or mechanical polishing, thereby retaining hydrogel in the wells but removing or inactivating substantially all of the hydrogel from the interstitial regions on the surface of the structured substrate between the wells. The azido groups of the hydrogel material may be converted to, or coupled to, groups with a positive charge. A solution including the present particles may then be contacted with the polished substrate such that individual target particles may become attracted to, and disposed within, individual wells via interactions with the positively charged groups attached to the hydrogel material; however, the particles substantially will not occupy the interstitial regions due to absence or inactivity of the hydrogel material. Before or after contacting the particles with the substrate, the particles may be used to capture and amplify a plurality of target polynucleotides (e.g., a fragmented human genome or portion thereof). Amplicons of the target polynucleotides may be confined to the wells because the particles are confined to the wells. The amplicons then may be sequenced. The process is conveniently manufacturable, being scalable and utilizing conventional micro- or nano-fabrication methods.

A patterned substrate may include, for example, wells etched into a slide or chip. The pattern of the etchings and geometry of the wells may take on a variety of different shapes and sizes, and such features may be physically or functionally separable from each other. Particularly useful substrates having such structural features include patterned substrates that may select the size of solid particles such as microspheres. An exemplary patterned substrate having these characteristics is the etched substrate used in connection with BEAD ARRAY technology (Illumina, Inc., San Diego, Calif.).

In some examples, a substrate described herein forms at least part of a flowcell or is located in or coupled to a flowcell. Flowcells may include a flow chamber that is divided into a plurality of lanes or a plurality of sectors. Example flowcells and substrates for manufacture of flowcells that may be used in methods and compositions set forth herein include, but are not limited to, those commercially available from Illumina, Inc. (San Diego, CA).

As used herein, a “hydrogel” refers to a three-dimensional polymer network structure that includes polymer chains and is at least partially hydrophilic and contains water within spaces between the polymer chains. A hydrogel may include any suitable combination of hydrophilic, hydrophobic, and/or amphiphilic polymer(s), so long as the overall polymer network is hydrophilic and contains water within spaces between the polymer chains. Hydrogels include chemical hydrogels in which both the bonding to form the polymer chains, and any cross-linking between the polymer chains, is covalent; such cross-linking during hydrogel formation may be irreversible, as distinguished from the present reversible cross-linking which is performed after the hydrogel is formed. In some cases, the chemical hydrogel may include, or may consist essentially of, brush-like structures of polymer chains attached to a surface, substantially without physical or covalent crosslinks between polymer chains, or alternatively polymer chains with multiple attachment points to a surface, resulting in loops, but also lacking interchain crosslinks. Hydrogels also include physical hydrogels in which the bonding to form the polymer chains, and any cross-linking within the polymer chains, is not covalent. Nonlimiting examples of physical hydrogels include agarose and alginate. A hydrogel may be located on a substrate, such as on a particle or on a region of a flowcell.

As used herein, the “polymer chain” of a hydrogel is intended to mean those portions of the hydrogel that are polymerized with one another during the polymerization process. Polymer chains may be cross-linked to form the hydrogel. For example, cross-linkers may be added during or after the polymerization process that forms the polymer chains. Additionally, or alternatively, in some examples the polymer chains may be deposited on a substrate surface that includes functional groups to which functional groups of the polymer chains become coupled. The polymer chains may be coupled to the surface, e.g., via reactions between the functional groups of the polymer chains and the functional groups at the surface, and such coupling may cross-link the polymer chains to form the hydrogel. Such cross-linking may cause the polymer chains to covalently or non-covalently attach to one another, or may occur as a result of chain entanglement during polymerization and/or attachment to a surface.

As used herein, the term “directly” when used in reference to a layer covering the surface of a substrate is intended to mean that the layer covers the substrate's surface without a significant intermediate layer, such as, e.g., an adhesive layer or a polymer layer. Layers directly covering a surface may be attached to this surface through any chemical or physical interaction, including covalent bonds or non-covalent adhesion.

As used herein, the term “immobilized” when used in reference to a polynucleotide is intended to mean direct or indirect attachment to a substrate via covalent or non-covalent bond(s). In certain examples, covalent attachment may be used, or any other suitable attachment in which the polynucleotides remain stationary or attached to a substrate under conditions in which it is intended to use the substrate, for example, in polynucleotide amplification or sequencing. Polynucleotides to be used as capture primers or as target polynucleotides may be immobilized such that a 3′-end is available for enzymatic extension and at least a portion of the sequence is capable of hybridizing to a complementary sequence. Immobilization may occur via hybridization to a surface attached oligonucleotide, in which case the immobilized oligonucleotide or polynucleotide may be in the 3′-5′ orientation. Alternatively, immobilization may occur by means other than base-pairing hybridization, such as covalent attachment.

As used herein, the term “array” refers to a population of substrate regions that may be differentiated from each other according to relative location. Different molecules (such as polynucleotides coupled to respective particles) that are at different regions of an array may be differentiated from each other according to the locations of the regions in the array. An individual region of an array may include one or more molecules of a particular type. For example, a substrate region may include a single particle including amplicons of a target polynucleotide having a particular sequence. The regions of an array respectively may include different features than one another on the same substrate. Exemplary features include without limitation, wells in a substrate, particles (such as beads) in or on a substrate, projections from a substrate, ridges on a substrate or channels in a substrate. The regions of an array respectively may include different regions on different substrates than each other. Different molecules attached to separate substrates may be identified according to the locations of the substrates on a surface to which the substrates are associated or according to the locations of the substrates in a liquid or hydrogel. Exemplary arrays in which separate substrates are located on a surface include, without limitation, those having particles (such as beads) in wells.

As used herein, the term “plurality” is intended to mean a population of two or more different members. Pluralities may range in size from small, medium, large, to very large. The size of small plurality may range, for example, from a few members to tens of members. Medium sized pluralities may range, for example, from tens of members to about 100 members or hundreds of members. Large pluralities may range, for example, from about hundreds of members to about 1000 members, to thousands of members and up to tens of thousands of members. Very large pluralities may range, for example, from tens of thousands of members to about hundreds of thousands, a million, millions, tens of millions and up to or greater than hundreds of millions of members. Therefore, a plurality may range in size from two to well over one hundred million members as well as all sizes, as measured by the number of members, in between and greater than the above exemplary ranges. Exemplary polynucleotide pluralities include, for example, populations of about 1×10⁵ or more, 5×10⁵ or more, or 1×10⁶ or more different polynucleotides. Accordingly, the definition of the term is intended to include all integer values greater than two. An upper limit of a plurality may be set, for example, by the theoretical diversity of polynucleotide sequences in a sample.

As used herein, the term “double-stranded,” when used in reference to a polynucleotide, is intended to mean that all or substantially all of the nucleotides in the polynucleotide are hydrogen bonded to respective nucleotides in a complementary polynucleotide. A “partially” double stranded polynucleotide may have at least about 10%, at least about 25%, at least about 50%, at least about 60%, at least about 70%, at least about 80%, at least about 90% or at least about 95% of its nucleotides, but fewer than all of its nucleotides, hydrogen bonded to nucleotides in a complementary polynucleotide.

As used herein, the term “single-stranded,” when used in reference to a polynucleotide, means that essentially none of the nucleotides in the polynucleotide are hydrogen bonded to a respective nucleotide in a complementary polynucleotide. A polynucleotide that has an “inability” to hybridize to another polynucleotide may be single-stranded.

As used herein, the term “target polynucleotide” is intended to mean a polynucleotide that is the object of an analysis or action. The analysis or action includes subjecting the polynucleotide to amplification, sequencing and/or other procedure. A target polynucleotide may include nucleotide sequences additional to a target sequence to be analyzed. For example, a target polynucleotide may include one or more adapters, including an adapter that functions as a primer binding site, that flank(s) a target polynucleotide sequence that is to be analyzed. A target polynucleotide hybridized to a capture primer may include nucleotides that extend beyond the 5′ or 3′ end of the capture oligonucleotide in such a way that not all of the target polynucleotide is amenable to extension. In particular examples, target polynucleotides may have different sequences than one another but may have first and second adapters that are the same as one another. The two adapters that may flank a particular target polynucleotide sequence may have the same sequence as one another, or complementary sequences to one another, or the two adapters may have different sequences. Thus, species in a plurality of target polynucleotides may include regions of known sequence that flank regions of unknown sequence that are to be evaluated by, for example, sequencing (e.g., SBS). In some examples, target polynucleotides carry an adapter at a single end, and such adapter may be located at either the 3′ end or the 5′ end the target polynucleotide. Target polynucleotides may be used without any adapter, in which case a primer binding sequence may come directly from a sequence found in the target polynucleotide.

The terms “polynucleotide” and “oligonucleotide” are used interchangeably herein. The different terms are not intended to denote any particular difference in size, sequence, or other property unless specifically indicated otherwise. For clarity of description the terms may be used to distinguish one species of polynucleotide from another when describing a particular method or composition that includes several polynucleotide species.

As used herein, the term “amplicon,” when used in reference to a polynucleotide, is intended to means a product of copying the polynucleotide, wherein the product has a nucleotide sequence that is substantially the same as, or is substantially complementary to, at least a portion of the nucleotide sequence of the polynucleotide. “Amplification” and “amplifying” refer to the process of making an amplicon of a polynucleotide. A first amplicon of a target polynucleotide may be a complementary copy. Additional amplicons are copies that are created, after generation of the first amplicon, from the target polynucleotide or from the first amplicon. A subsequent amplicon may have a sequence that is substantially complementary to the target polynucleotide or is substantially identical to the target polynucleotide. It will be understood that a small number of mutations (e.g., due to amplification artifacts) of a polynucleotide may occur when generating an amplicon of that polynucleotide.

A substrate region that includes substantially only amplicons of a given polynucleotide may be referred to as “monoclonal,” while a substrate region that includes amplicons of polynucleotides having different sequences than one another may be referred to as “polyclonal.” A substrate region that includes a sufficient number of amplicons of a given polynucleotide to be used to sequence that polynucleotide maybe referred to as “functionally monoclonal.” Illustratively a substrate region in which about 60% or greater of the amplicons are of a given polynucleotide may be considered to be “functionally monoclonal.” Additionally, or alternatively, a substrate region from which about 60% or more of a signal is from amplicons of a given polynucleotide may be considered to be “functionally monoclonal.” A polyclonal region of a substrate may include different subregions therein that respectively are monoclonal. Each such monoclonal region, whether within a larger polyclonal region or on its own, may correspond to a “cluster” generated from a “seed.” The “seed” may refer to a single target polynucleotide, while the “cluster” may refer to a collection of amplicons of that target polynucleotide. The action by which a polynucleotide in solution becomes coupled to a substrate may be referred to as “capturing” the polynucleotide.

As used herein, the term “magnetic” is intended to refer to a material or combination of materials that is attracted to an applied magnetic field. Magnetic materials include ferromagnetic materials (such as materials including iron, cobalt, nickel, or a combination of any of such materials with one another and/or with a material which is not ferromagnetic). Accordingly, a “magnetic particle” referred to herein includes a particle that, when disposed in a liquid and exposed to a sufficiently high external magnetic field, can move through the liquid due to attraction to the source of the external magnetic field.

### Methods of Functionalizing Magnetic Particles

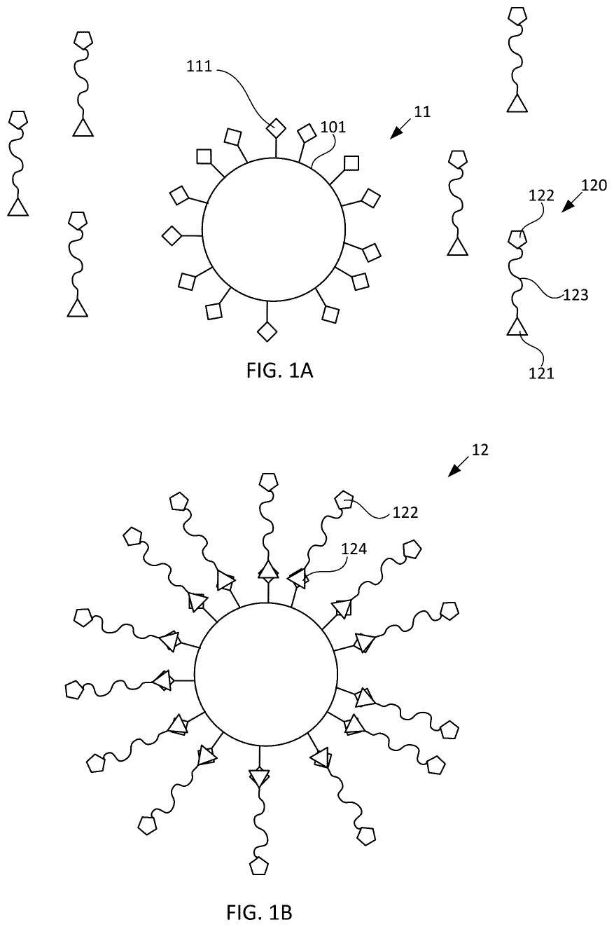

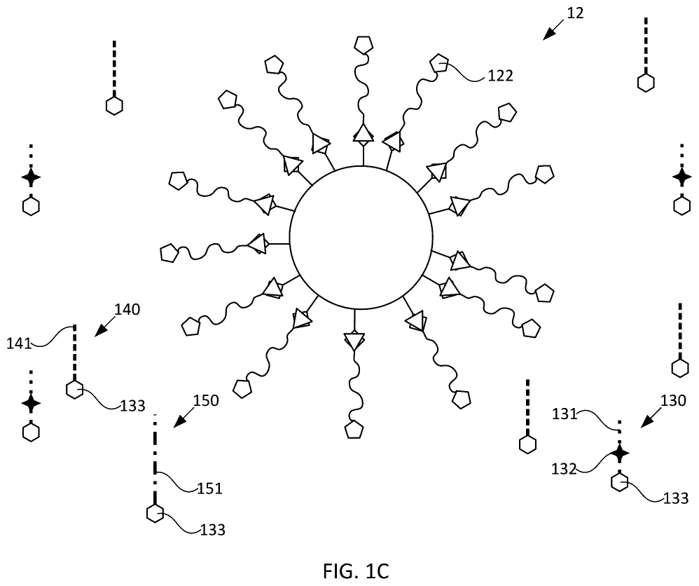

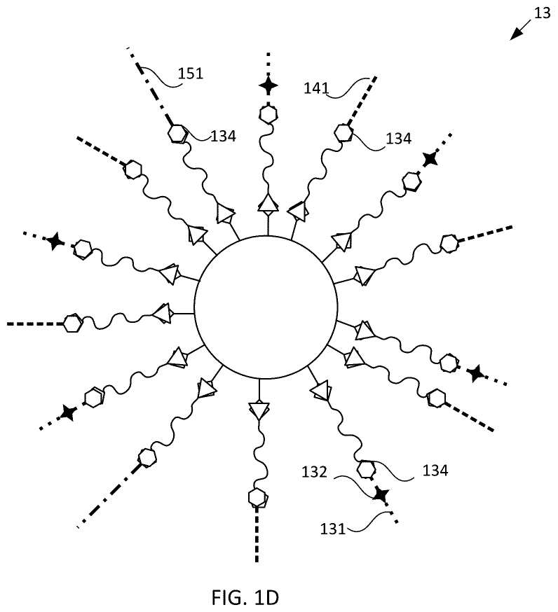

Some examples herein provide methods of functionalizing magnetic particles, and particles where are prepared using such methods. For examples, FIGS. 1A-1D schematically illustrate example structures and operations for preparing a magnetic particle coupled to a polymer, and optionally also to oligonucleotides. Referring now to FIG. 1A, the present magnetic particles may be prepared using a starting magnetic particle 11 that includes a core 101 the outer surface of which includes a plurality of first functional groups 111. Magnetic particles that include a variety of functional groups are commercially available. Optionally, particle 11 may have a diameter size ranging from about 10 nm to about 1 μm, e.g., about 50 nm to about 500 nm, e.g., about 100 nm to about 300 nm, or about 250 nm to about 300 nm, with low polydispersity index (PDI<1) (ISO 22,412:2017).

In some examples, the magnetic particles are contacted with molecules that contain a second functional group and a third functional group. For example, as illustrated in FIG. 1A, preparing a magnetic particle may include contacting particle 11 with second molecules 120. Each of the second molecules 112 may include a polymer 123 coupled to a second functional group 121 and a third functional group 122. Although the second functional group 121 and third functional group 122 are illustrated for simplicity as being located at respective ends of polymer 123, it should be appreciated that these functional groups may have any suitable location relative to one another and relative to the polymer. In some examples, the polymer may include a polyacrylamide, such as PAZAM or SFA, that is synthesized in a manner as to include functional groups 121 and 122. Alternatively, the functional groups may be coupled to the polymer in a separate operation. In some examples, functional groups 121 and 122 may be of the same type as one another, while in other examples, functional groups 121 and 122 may be of different types than one another. Functional group 121 may be a partner pair to functional group 111. For example, functional group 111 may include a norbornene, a cyclooctyne, or a bicyclononyne, and functional group 121 may include an azide. For nonlimiting examples of pairs of functional groups which may be used to couple a surface to a polymer, see U.S. Pat. No. 10,975,210 to Berti et al., the entire contents of which are incorporated by reference herein. Functional group 122 may be of the same type of group as functional group 121, or may be of a different type of group. For nonlimiting examples of polymers including functional groups, see U.S. Pat. No. 10,975,210. FIG. 1B illustrates particle 12 generated by reaction of particle 11 with a plurality of second molecules 120. Particle 12 includes second functional groups 121 and third functional groups 122 coupled to core 110 via polymer 123, via the reaction product 124 resulting from reaction of first functional group 111 and second functional group 121.

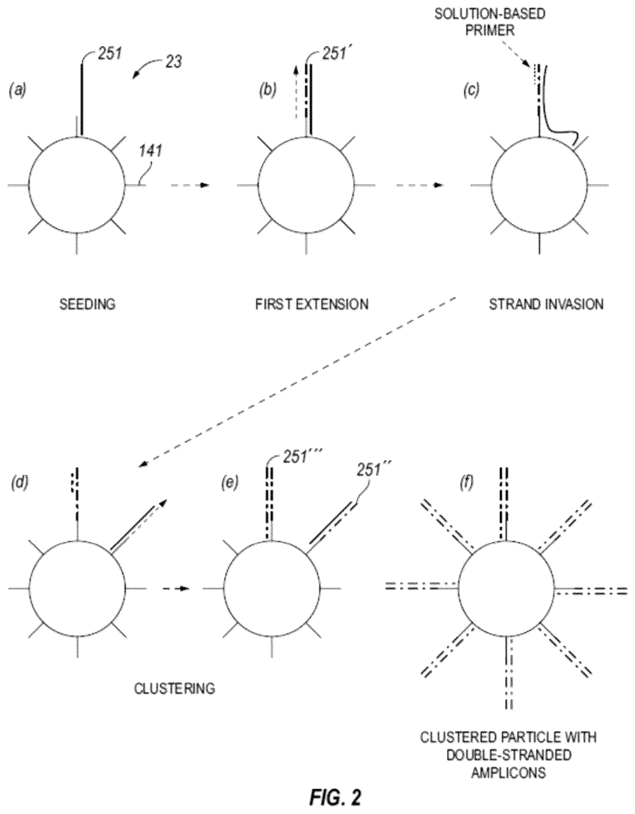

In some examples, oligonucleotides may be coupled to magnetic particle 12, e.g., so as to form a functionalized magnetic particle that optionally may be used in operations such as will be described with reference to FIGS. 2A-2H. For example, as illustrated in FIG. 1C, particle 12 may be contacted with third molecules each of which may include an oligonucleotide coupled to a fourth functional group 133. In the nonlimiting example illustrated in FIG. 1C, particle 12 is contacted with at least one of third molecules 130, 140, and/or 150, and optionally with a mixture of third molecules 130, 140, and/or 150. Molecules 130 may include a first oligonucleotide 131, which is coupled to fourth functional group 133 and optionally includes an excision moiety 132. Molecules 140 may include a second oligonucleotide 141 which is coupled to fourth functional group 133. Molecules 150 may include a third oligonucleotide 151 which is coupled to fourth functional group 133. The sequences of oligonucleotides 131, 141, and 151 may be orthogonal to one another. Illustratively, oligonucleotide 131 may be an amplification primer having the sequence P5 described further below, oligonucleotide 141 may be a different amplification primer having the sequence P7 described further below, and oligonucleotide 151 may be a capture primer having the sequence PX or PY described further below. FIG. 1D illustrates particle 13 generated by reaction of particle 12 with a plurality of third molecules, e.g., molecules 130, or molecules 140, or molecules 150, or a mixture of molecules 130 and 140, or a mixture of molecules 130 and 150, or a mixture of molecules 140 and 150, or a mixture of molecules 130, 140, and 150. As such, particle 13 includes oligonucleotides 131, 141, and/or 151 respectively coupled to core 101 via the reaction products 134 between third functional group 133 and second functional group 122, via polymer 123, via the reaction product 124 resulting from reaction of first functional group 111 and second functional group 121 in a preceding operation.

It will be appreciated that any suitable moieties may be used to couple elements to one another to form particle 13, e.g., to couple moiety 122 to moiety 133 to form reaction product 134. For example, such elements may be coupled to one another using covalent bonding selected from the group consisting of azide-alkyne bonding, transcyclooctene-tetrazine bonding, norbornene-tetrazine bonding, azide-cyclooctyne bonding, and azide-norbornene bonding.

A non-exclusive list of complementary binding partners that may be used to covalently couple moiety 122 to moiety 133 to form reaction product 134 is presented in Table 1. Note that moiety 121 and 122 can be of the same type as one another in certain examples, as explained above:

TABLE 1

| Bonding pair | Example moiety 122 | Example moiety 133 |
|---|---|---|
| azide-alkyne | azide, —N₃ | alkyne |
| | | 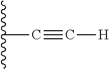 [SMILES: *C#C] |
| azide-cyclooctyne or azide- | azide, —N₃ | cyclooctyne, e.g. dibenzocyclooctyne |
| cyclononine | | (DBCO) |
| | | 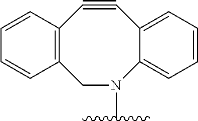 [SMILES: *N1Cc2ccccc2C#Cc2ccccc21] |
| | | or BCN (bicyclo[6.1.0]nonyne) |
| | | 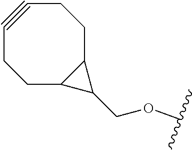 [SMILES: *OCC1C2CCC#CCCC21] |
| azide-norbornene | azine, —N₃ | norbornene |
| | | 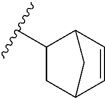 [SMILES: *C1CC2C=CC1C2] |
| transcyclooctene-tetrazine | tetrazine, e.g., benzyl- | transcyclooctene |
| | methyltetrazine | |
| | 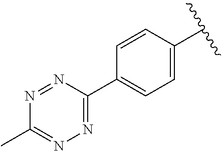 [SMILES: *c1ccc(-c2nnc(C)nn2)cc1] | 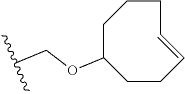 [SMILES: *COC1CC/C=C/CCC1] |
| norbornene-tetrazine | tetrazine, e.g., benzyl- | norbornene |
| | methyltetrazine | |
| | 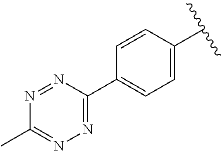 [SMILES: *c1ccc(-c2nnc(C)nn2)cc1] | 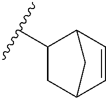 [SMILES: *C1CC2C=CC1C2] |

It is believed that particles 13 may be particularly well suited for use in capturing and amplifying polynucleotides, e.g., to generate substantially monoclonal clusters in a manner such as will be described below with reference to FIGS. 2A-2H. For example, the particles 13 may be used to generate the clusters in solution, which may facilitate the resulting clusters being substantially monoclonal. Additionally, the particles may be readily collected and/or washed without the need for filtration or centrifugation, by applying an external magnetic field that attracts the particles out of solution and into a location where they can be collected, and the wash liquid discarded. Additionally, it is believed that particles 13 may be particularly well suited for use in sequencing the amplified polynucleotides on a flowcell, in a manner that allows for the flowcell to be easily reused.

### Methods for Capturing Polynucleotides on Magnetic Particles

Some examples herein provide a method of generating an amplicon that includes capturing a polynucleotide with a magnetic particle. The magnetic particle may include a plurality of amplification primers that may be used to generate amplicons of the captured polynucleotide.

FIG. 2 schematically illustrates an example of a workflow for generating amplicons using a magnetic particle. Referring now to operation (a) illustrated in FIG. 2, magnetic particle 23 includes a plurality of amplification primers 141 which may be coupled to the particle in a manner such as described with reference to FIGS. 1A-ID (e.g., via a polymer). Magnetic particle 23 illustrated in FIG. 2A may correspond to magnetic particle 12 described with reference to FIG. 1D, and may be formed using operations such as described with reference to FIGS. 1A-1D. In this example, magnetic particles 23 include only a single type of amplification primer 141 (illustratively, P7), rather than a mix of different amplification primers. Additionally, in this example, magnetic particles 23 do not include any seeding primers that have a different sequence than primer 141. Instead, one or more of amplification primers 141 are used both to capture a polynucleotide and to amplify that polynucleotide. Particle 23 is contacted with a single-stranded polynucleotide 251 which includes a first adapter which is complementary to, and which hybridizes to, amplification primer 141; and a second adapter which has at least partially the same sequence as a solution-based primer which will be described further below. Referring now to operation (b) illustrated in FIG. 2, the primer 141 to which the first adapter of polynucleotide 251 hybridizes is extended, for example using a polymerase, to form an amplicon 251′ which is covalently coupled to the particle via the extended amplification primer 141.

A strand invasion process then is used to generate additional amplicons of polynucleotide 251. For example, referring now to operation (c) illustrated in FIG. 2, a solution-based primer, illustratively a truncated P5 primer having at least partially the same sequence as the second adapter of polynucleotide 251, hybridizes to the second adapter of amplicon 251′ using strand invasion which is promoted by a recombinase. Additionally, the first adapter of polynucleotide 251 hybridizes to another amplification primer 141 using strand invasion which is promoted by a recombinase. As illustrated in operation (d) of FIG. 2, the second primer 141 to which the first adapter of polynucleotide 251 hybridizes is extended, for example using a polymerase, to form a second amplicon 251″ which is covalently coupled to the particle via the extended amplification primer 141. Additionally, the solution-based primer which is hybridized to amplicon 251′ is extended, for example using a polymerase, to form a third amplicon 251′″ which is not covalently coupled to the particle, but rather hybridized to first amplicon 251′, as illustrated in operation (e) of FIG. 2. As illustrated in operation (f) of FIG. 2, the polynucleotides which are not covalently coupled to the particle, e.g., amplicon 251′″ and polynucleotide 251, then may be further amplified using strand invasion in a manner such as described with reference to operations (c), (d), and (c) of FIG. 2 to generate a clustered particle with double-stranded amplicons. For further details of amplification processes such as may be used during, and/or are compatible with, operations such as described with reference to FIG. 2, see International Patent Application No. PCT/US2022/053005 to Ma et al., filed Dec. 15, 2022 and entitled “Hybrid Clustering,” and International Patent Application No. PCT/EP2023/058307 to Ma et al., filed Mar. 30, 2023 and entitled “Paired-End Resynthesis Using Blocked P5 Primers,” the entire contents of each of which are incorporated by reference herein. At any suitable time during operations such as described with reference to FIG. 2, an external magnetic field may be applied so as to attract the particles out of solution such that they may be washed, and any other solution components may be discarded.

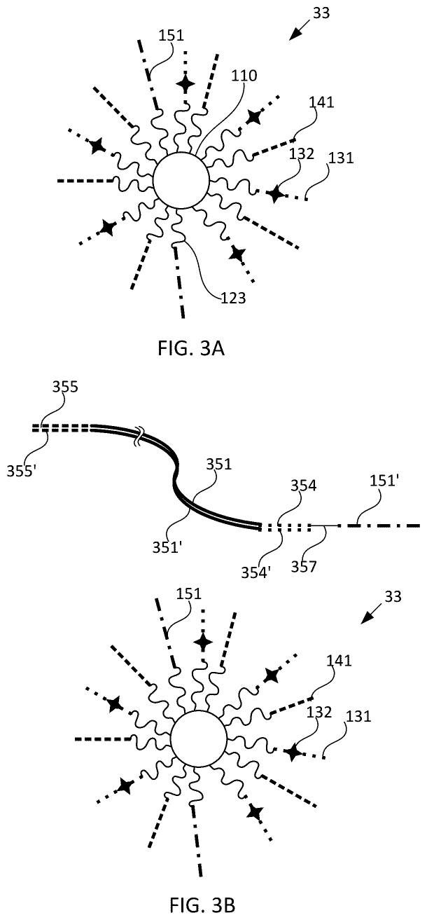

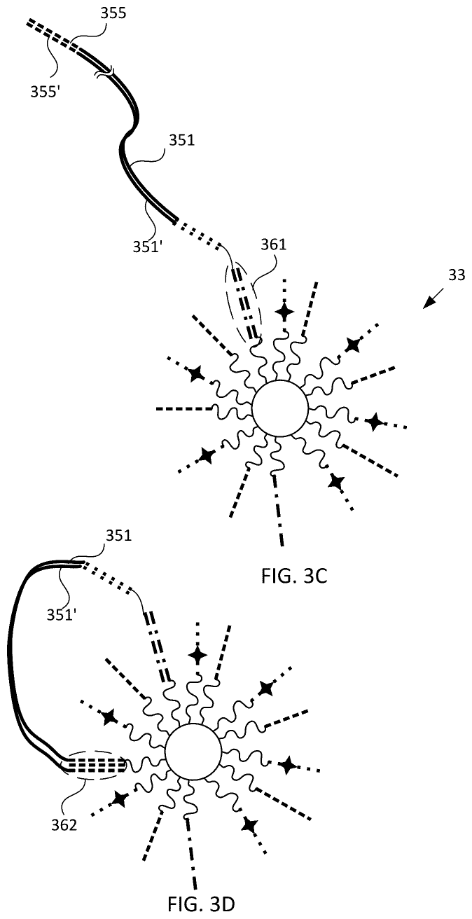

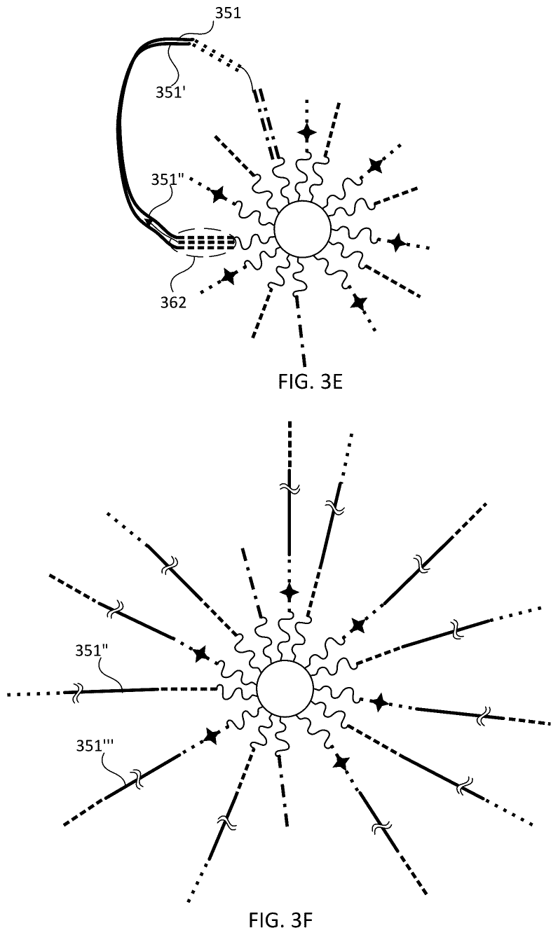

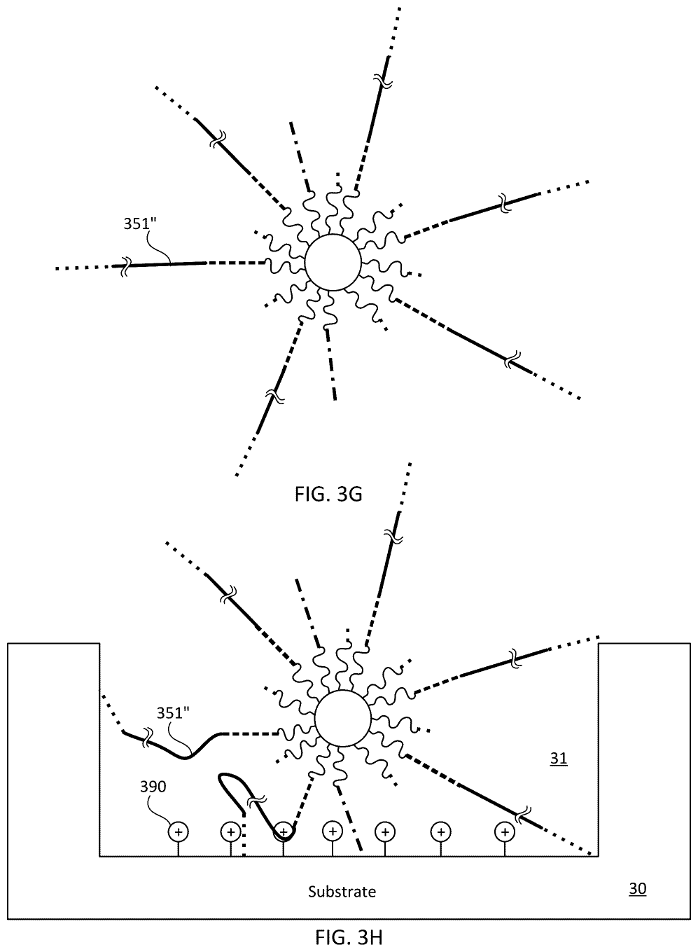

It will be appreciated that the present magnetic particles may be used with other amplification operations to generate amplicons. For example, FIGS. 3A-3H schematically illustrate an alternative example workflow for generating amplicons using a magnetic particle. Referring to FIG. 3A, magnetic particle 33 may include a plurality of each of first and second amplification primers 131, 141 which have orthogonal sequences to one another. Magnetic particle 33 illustrated in FIG. 3A may correspond to magnetic particle 12 described with reference to FIG. 1D, and may be formed using operations such as described with reference to FIGS. 1A-1D. In the nonlimiting example illustrated in FIG. 3A, magnetic particle 33 may include one or more seeding primers 151 which have an orthogonal sequence to that of amplification primers 131, 141 and which may be used to capture a polynucleotide having an adapter which is complementary to seeding primer 151 in a manner such as will be described with reference to FIGS. 3B-3C. In other examples, particle 33 may not include any seeding primers, and a sub-Poisson approach may be used to seed particles 33. More specifically, magnetic particles 33 may be contacted with a solution which contains a sufficiently dilute concentration of polynucleotides that each particle 33 is statistically likely to capture either one or zero polynucleotides from the solution. Magnetic particles 33 which capture one polynucleotide may then be used to amplify that polynucleotide to generate a cluster of amplicons, e.g., using bridge amplification, exclusion amplification (ExAmp), or other suitable amplification method, while a cluster of amplicons is not generated on particles which did not capture a polynucleotide. The magnetic particles with a cluster of amplicons have a different size, and a different charge, than particles without a cluster of amplicons. As such, the magnetic particles with the cluster of amplicons may be separated via enrichment based on size and/or charge from the particles without a cluster of amplicons.

At the particular time illustrated in FIG. 3A, magnetic particle 33 may be located within a fluid (fluid not specifically illustrated), such an aqueous buffer solution. In the nonlimiting example illustrated in FIG. 3B, magnetic particle 33 may be contacted, in the fluid, with polynucleotides, illustratively double-stranded target polynucleotides, e.g., double-stranded polynucleotide fragments that are generated using commercially available fragmentation or tagmentation techniques, using DNA or RNA that it is desired to sequence. Although only one magnetic particle 33 is illustrated in FIG. 3B, it will be appreciated that the fluid may include thousands, or even millions, of magnetic particles that have substantially the same configuration as one another. Additionally, although only one double-stranded polynucleotide 351, 351′ is illustrated in FIG. 3B, it will be appreciated that the fluid may include thousands, or even millions, of polynucleotides which in some cases may have different lengths than one another.

Some examples herein provide a method of generating an amplicon that includes coupling to a magnetic particle a first double-stranded polynucleotide including first and second strands hybridized to one another. In some examples, the first strand includes a first amplification adaptor. In some examples, the second strand includes a second amplification adaptor that is complementary to the first amplification adaptor.

For example, as illustrated in FIG. 3B, a double-stranded polynucleotide includes a first strand 351 and second strand 351′ that are hybridized to each other. The first strand 351 contains a first amplification adaptor 354. The second strand 351′ contains a second amplification adaptor 354′. The first amplification adaptor 354 and the second amplification adaptor 354′ are complementary to each other.

Accordingly, each of the polynucleotides 351, 351′ may include first and second double-stranded amplification adapters, e.g., first adapter 354, 354′ one strand of which is substantially complementary to amplification primer 131, e.g., is or includes cP5 (also referred to as P5′); and second adapter 355, 355′ one strand of which is substantially complementary to amplification primer 141, e.g., is or includes cP7 (also referred to as P7′). The double-stranded polynucleotides optionally may include an additional single-stranded seeding adapter which may be used to immobilize the polynucleotide to the particle during the seeding process. For example, as shown in FIG. 3B, polynucleotide 351, 351′ optionally may include single-stranded seeding adapter 151′ which is substantially complementary to seeding primer(s) 151, and which optionally is coupled to amplification adaptor 354 of polynucleotide 351 via linker 357. In some examples, optional seeding adapter 151′ may include cPX (also referred to as PX′) or cPY (also referred to as PY′). Note that because it is single-stranded, optional seeding adapter 151′ is available to hybridize to optional seeding primer 151 in a manner such as now will be described. Other methods for capture a polynucleotide using amplification primers 131, 141 (e.g., sub-Poisson methods) without the use of seeding primers and seeding adapters suitably may be used.

In nonlimiting examples including use of seeding primer 151 and seeding adapter 151′, optional linker 357 may be or include one or more of: a carbon-containing chain with a formula (CH₂)ₙ wherein “n” is from 1 to about 1500, for example less than about 1000, preferably less than 100, e.g. from 2-50, particularly 5-25; polyethylene glycol (PEG); or iSp9 (Spacer 9) which is a triethylene glycol spacer that can be incorporated at the 5′-end or 3′-end of an oligo or internally. Linkers formed primarily from chains of carbon atoms or from PEG may be modified so as to contain functional groups which interrupt the chains. Examples of such groups include ketones, esters, amines, amides, ethers, thioethers, sulfoxides, sulfones. Separately or in combination with the presence of such functional groups, groups such as alkene, alkyne, aromatic or heteroaromatic moieties, or cyclic aliphatic moieties (e.g., cyclohexyl) may be included. Cyclohexyl or phenyl rings may, for example, be connected to a PEG or (CH₂)ₙ chain through their 1- and 4-positions. Other linkers are envisaged which are based on nucleic acids or monosaccharide units (e.g., dextrose). It is also within the scope of this disclosure to utilise peptides as linkers. A variety of other linkers may be employed. The linker preferably is stable under conditions under which the polynucleotides are intended to be used subsequently, e.g., conditions used in DNA amplification. The linker should also be such that it is not by-passed by DNA polymerases, terminating DNA polymerization before copying optional adapter 151′ (if it is nucleotide based such as a PX′ sequence). This allows for the optional adapter 151′ to remain single-stranded and available for hybridization in a manner such as will now be described.

Referring now to the nonlimiting example shown in FIG. 3C, seeding adapter 151′ of double-stranded polynucleotide 351, 351′ may hybridize to seeding primer 151 to form duplex 361. Because amplification adaptors 354, 354′ and 355, 355′ are double-stranded, they are not available to hybridize to primers 131 and 141 respectively until after certain processing steps are performed. Accordingly, substantially all seeding of the polynucleotide may be expected to be via hybridization between seeding adapter 151′ and seeding primer 151.

Referring now to FIG. 3C, seeding adapter 151′ of double-stranded polynucleotide 351, 351′ may hybridize to seeding primer 151 to form duplex 361. Because adapters 354, 354′ and 355, 355′ are double-stranded, they are not available to hybridize to adapters 131 and 141 respectively until after certain processing steps are performed, as will be explained below. Accordingly, substantially all seeding of the polynucleotide may be expected to be via hybridization between seeding adapter 151′ and seeding primer 151.

For example, after the initial hybridization described with reference to FIG. 3C, polynucleotide 351, 351′ may be amplified relatively quickly. More specifically, as illustrated in FIG. 3D, the double-stranded polynucleotide 351, 351′ may bend such that adapter 355, 355′ hybridizes with one of primers 141 to form triplex 362 using a process that may be referred to as “strand invasion” and may be promoted using a recombinase (not specifically illustrated in FIG. 3D). An amplified cluster then may be formed using polynucleotide 351, 351′. For example, FIG. 3E illustrates the composition of FIG. 3D during recombinase-promoted extension of the primer 341 to which double-stranded polynucleotide 351, 351′ hybridizes to form amplicon 351″. Amplicon 351″ repeatedly may be further amplified using strand invasion. For example, it may be seen that the composition of FIG. 3F includes amplicon 351″ and a plurality of additional amplicons 351′″ of amplicon 351″ that are formed using a mixture of capture primers 131 and 141.

Amplification operations may be performed any suitable number of times so as to substantially fill the particle with an at least functionally monoclonal cluster, and in some examples a substantially monoclonal cluster, e.g., with amplicons of target polynucleotide 351, 351′. For example, amplicons within the second region of the particle may include at least about 60% amplicons of one selected target polynucleotide, or at least about 70% amplicons of one selected target polynucleotide, or at least about 80% amplicons of one selected target polynucleotide, or at least about 90% amplicons of one selected target polynucleotide, or at least about 95% amplicons of one selected target polynucleotide, or at least about 98% amplicons of one selected target polynucleotide, or at least about 99% amplicons of one selected target polynucleotide, or about 100% amplicons of one selected target polynucleotide. If amplification operations are repeated until the second region of the particle is substantially full, both adapters of the resulting amplicons may not necessarily be hybridized to corresponding capture primers or orthogonal capture primers, and as such the amplicons may extend linearly away from the particle as illustrated in FIG. 3F. For further details regarding seeding and amplification operations using strand invasion and seeding adapters and seeding primers that are orthogonal to capture adapters and capture primers, see International Patent Application No. PCT/US2022/053002, filed on Dec. 15, 2022 and entitled “Orthogonal Hybridization,” the entire contents of which are incorporated by reference herein.

In some examples, certain capture primers and orthogonal capture primers may include non-nucleotide moieties. Such non-nucleotide moieties may include, but are not limited to, excision moieties via which a portion of the capture primers selectively may be removed. For example, capture primers 131 optionally may include excision moieties 132 and/or capture primers 141 optionally may include excision moieties (excision moieties of capture primers 141 not specifically shown in FIGS. 3A-3F). The excision moieties 132 of capture primers 131 may be of the same type as, or a different type than, the excision moieties of capture primers 141. The excision moieties may be located at any suitable position along the length of any suitable primer(s) and may be, but need not necessarily be, the same type of excision moiety as one another. Following a desired number of amplification operations such as described with reference to FIGS. 3A-3F, portions of capture primers 131 may be removed by reacting a suitable enzyme or reagent with the respective excision moieties, and/or portions of capture primers 141 may be removed by reacting a suitable enzyme or reagent with the respective excision moieties. The enzyme or reagent used with the excision moieties of capture primers 131 may be the same as, or different than, the enzyme or reagent used with excision moieties of capture primers 141. Excision moieties 132 may be used to remove amplicons that are oriented in a selected direction, so that the remaining amplicons are oriented substantially in the same direction as one another in a manner such as illustrated in FIG. 3G. At any suitable time during operations such as described with reference to FIGS. 3A-3G, an external magnetic field may be applied so as to attract the particles out of solution such that they may be washed, and any other solution components may be discarded.

In some examples, following the seeding and amplification operations, the magnetic particle with amplicons 351″ coupled thereto may be disposed within a flowcell and the amplicons sequenced, e.g., using sequencing-by-synthesis. Illustratively, as shown in FIG. 3H, a substrate 30 (e.g., a surface of a flowcell) may include a plurality of positively charged moieties 390. In the nonlimiting example illustrated in FIG. 3H, the substrate 30 includes a recess 31 in which the positively charged moieties 390 are disposed. Optionally, the positively charged moieties may be coupled to a hydrogel (not specifically illustrated) which is located on the substrate (e.g., within the flowcell). The amplicons 351″ may have a negative charge that is electrostatically attracted to the positively charged moieties 390. This electrostatic attraction may draw the magnetic particle into contact with the substrate. Additionally, this electrostatic attraction and/or other force(s) may retain the magnetic particle in contact with the substrate 10. The amplicons 351″ then may be sequenced, e.g., using sequencing-by-synthesis operations that use reagents which may be flowed across the substrate 30 (sequencing operations not specifically illustrated). Although not specifically described above, it will be appreciated that the clustered particles generated using the operations described with reference to FIG. 2 may be disposed within a flowcell. The amplicons which are not covalently coupled to the magnetic particle may be dehybridized, and the amplicons which are covalently coupled to the magnetic particle may be sequenced in a similar manner as described with reference to FIG. 3H, e.g., using sequencing-by-synthesis.

### Alternative Methods for Generating Amplicons Using Magnetic Particles

Although certain examples of using magnetic particles to generate amplicons are described with reference to FIGS. 2 and 3A-3H, it will be appreciated that magnetic particles may be used to capture polynucleotides, and to generate amplicons, in still other ways.

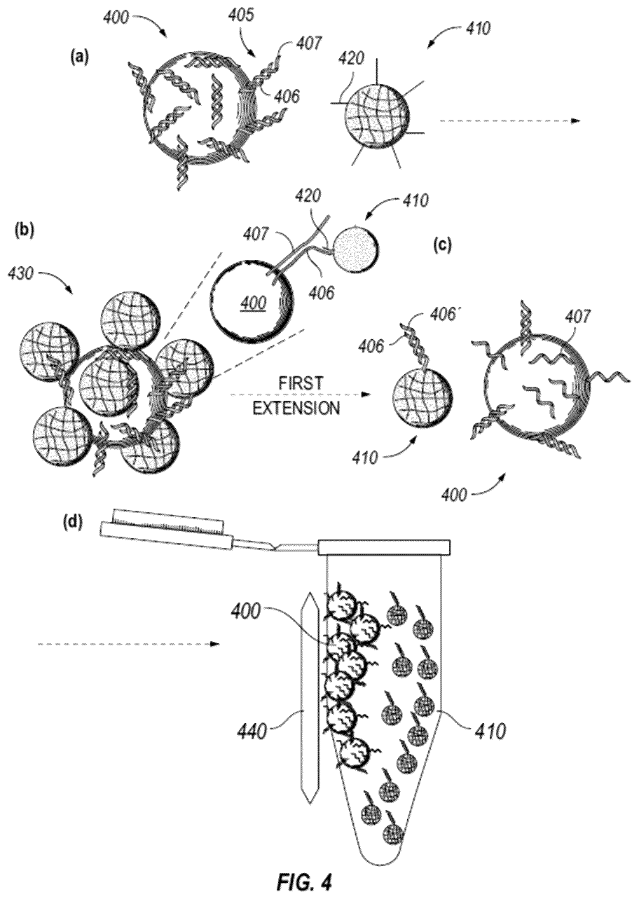

For example, FIG. 4 schematically illustrates an alternative example workflow for capturing a polynucleotide, and generating an amplicon, using a magnetic particle. Referring first to operation (a) illustrated in FIG. 4, first particle 400 may be coupled to a first double-stranded polynucleotide 405. Double-stranded polynucleotide 405 may include first and second strands 406, 407 hybridized to one another. First strand 406 may include a first amplification adaptor, and second strand 407 may include a second amplification adaptor that is complementary to the first amplification adapter. In some examples, first particle 400 may be magnetic, and in one specific example may include a solid phase reversible immobilization (SPRI) bead. SPRI beads are commercially available, e.g., from Beckman Coulter, Inc. In a manner such as known in the art, a library of polynucleotides may be prepared via tagmentation (or other suitable fragmentation technique which adds amplification adapters to the polynucleotide fragments), followed by size selection, enrichment, and purification using commercial SPRI beads. SPRI beads include carboxyl groups to which double-stranded polynucleotides adsorb. In the example illustrated in FIG. 4, particle 400 includes a plurality of double-stranded polynucleotides having different sequences than one another, each of which double-stranded polynucleotides may be configured similarly as double-stranded polynucleotide 405. In some examples, both strands of the double-stranded polynucleotide 405 are non-covalently attached (e.g., are adsorbed) to first particle 400.

Referring still to operation (a) illustrated in FIG. 4, first particle 400 may be contacted with second particle 410. Second particle 410 may be non-magnetic, and may include a hydrogel to which one or more first amplification primers 420 is coupled in a manner similar to that described with reference to FIGS. 1A-ID except that the particle is not magnetic. First amplification primer(s) 420 may be complementary to the first amplification adapter of first strand 406 of double-stranded polynucleotide 405. In some examples, first amplification primer(s) 420 correspond to primers 131 described with reference to FIGS. 1A-1D. Additionally, or alternatively, in some examples, first amplification primer(s) 420 correspond to primers 141 described with reference to FIGS. 1A-1D.

First amplification primer 420 may be hybridized to the first amplification adaptor of the first double-stranded polynucleotide using a recombinase-promoted strand-invasion process similar to that described with reference to FIGS. 2 and 3A-3H. For details regarding seeding and amplification operations using strand invasion, see International Patent Application No. PCT/US2022/053002, filed on Dec. 15, 2022 and entitled “Orthogonal Hybridization,” the entire contents of which are incorporated by reference herein. As illustrated in operation (b) of FIG. 4 and its inset, such strand invasion may couple second particle 410 to first particle 400. In examples illustrated in operation (b) of FIG. 4, a plurality of additional particles 410, each including one or more amplification primers 420, may become coupled to first particle 400 via a similar strand invasion of primers 420 to amplification adapters of other, respective double stranded polynucleotides 405, resulting in collection 430 including a plurality of particles 410 coupled to first particle 400. Any additional first amplification primers 420 that are coupled to particles 410 are omitted from the illustration for simplicity, but should be understood to be present.

In some examples, following strand invasion, the first amplification primer 420 of second particle 410 is extended to generate a first amplicon 406′ of the first strand 406 of the first double-stranded polynucleotide 405 such as illustrated in operation (c) of FIG. 4. For example, as shown in the inset of operation (b) in FIG. 4, the amplification primer 420 may be extended based on the sequence of first strand 406, e.g., using a polymerase, generating first amplicon 406′. This extension reaction releases second particle 410 from first particle 400. For example, first amplicon 406′ may be covalently coupled to particle via amplification primer 420, and may displace second strand 407 from hybridization to first strand 406. Because first strand 406 is non-covalently coupled to first particle 400 and non-covalently coupled to second particle 410, and is complementary to first amplicon 406′ which is covalently coupled to second particle 410, the strength of the hybridization between first strand 406 and first amplicon 406′ may detach first strand 406 from first particle 400, thus freeing second particle 410 from first particle 400. As illustrated in operation (c) of FIG. 4, the second strand 407 of polynucleotide 405 may remain non-covalently coupled to first particle 400, while a double-stranded polynucleotide including first strand 406 of polynucleotide 405 and first amplicon 406′ are coupled to second particle 410. Second particle(s) 410 may be separated from first particle(s) 400 in any suitable manner, for example using pull down. Illustratively, as illustrated in operation (d) of FIG. 4, a magnet 440 is used to attract the first particle(s) 400, leaving second particle(s) 410 in solution which can be removed (e.g., poured off), leaving the first particle(s) 400 behind. Other assays known the art can alternatively be used to separate first particle(s) 400 from second particle(s) 410.

The double-stranded polynucleotide 406″ coupled to particle 410 and including first strand 406 and first amplicon 406′ may be amplified on particle 410 in any suitable manner, e.g., in a manner such as described with reference to operations (c), (d), (c), and (f) of FIG. 2; in a manner such as described with reference to FIGS. 3D-3E; and/or in a manner such as described elsewhere herein. The resulting particle with plurality of amplicons coupled thereto then may be disposed in a flowcell in a manner such as described with reference to FIG. 3H, and the amplicons then sequenced, e.g., using sequencing-by-synthesis.

### Primers

From the foregoing disclosure, it will be appreciated that primers and adapters having any suitable sequences may be used. In one nonlimiting example, amplification primers 131 are P5 amplification primers, and amplification primers 141 are P7 amplification primers. P5 amplification primers, which are commercially available from Illumina, Inc. (San Diego, CA) have the sequence 5′-AATGATACGGCGACCACCGA-3′ (SEQ ID NO: 1). P7 amplification primers, which also are commercially available from Illumina, Inc., have the sequence 5′-CAAGCAGAAGACGGCATACGA-3′ (SEQ ID NO: 2). Adapters 154 may be full-length complementary P5 adapters (cP5) having the sequence 5′-TCGGTGGTCGCCGTATCATT-3′ (SEQ ID NO: 3), and are commercially available from Illumina, Inc. Adapters may be full-length complementary P7 adapters (cP7) having the sequence 5′-TCGTATGCCGTCTTCTGCTTG-3′ (SEQ ID NO: 4), and are commercially available from Illumina, Inc. Seeding primers 151 may have any suitable sequence which is orthogonal to the sequences of amplification primers 131, 141. In some examples, seeding primers 151 may be PX primers having the sequence AGGAGGAGGAGGAGGAGGAGGAGG (SEQ ID NO: 5). In other examples, seeding primers 151 may be PY primers having the sequence 5′-GAA GAA GAA GAA GAA GAA GAA GAA GAA GAA-3′ (SEQ ID NO: 6). In examples in which seeding primers 151 are PX primers, seeding adapters 151′ may be cPX (also referred to as PX′) adapters having the sequence CCTCCTCCTCCTCCTCCTCCTCCT (SEQ ID NO: 7). In examples in which seeding primers 151 are PY primers, seeding adapters 151′ may be cPY (also referred to as PY′) adapters having the sequence 5′-TTC TTC TTC TTC TTC TTC TTC TTC TTC TTC-3′ (SEQ ID NO: 8).

### Amplification Methods

It will be appreciated that various examples herein may be used with operations consistent with “bridge amplification” or “surface-bound polymerase chain reaction” and/or with other amplification modalities. One such amplification modality is “exclusion amplification,” or ExAmp. Exclusion amplification methods may further facilitate the amplification of a single target polynucleotide per particle and the production of a substantially monoclonal population of amplicons on a particle. For example, the rate of amplification of the first captured target polynucleotide using the present particles may be more rapid relative to much slower rates of transport and capture of target polynucleotides using the present particles. As such, the first target polynucleotide captured by the particle may be amplified rapidly and fill the entire particle, thus further inhibiting the seeding and/or amplification of additional target polynucleotide(s) by the particle. Even if a second target polynucleotide is captured at the particle after the first polynucleotide, the relatively rapid amplification of the first polynucleotide may fill enough of the particle to result in a signal that is sufficiently strong to perform sequencing by synthesis (e.g., the second region may be at least functionally monoclonal). The use of exclusion amplification may also result in super-Poisson distributions of particles including monoclonal clusters; that is, the fraction of particles in a collection of particles that are functionally monoclonal may exceed the fraction predicted by the Poisson distribution. Increasing super-Poisson distributions of useful clusters is useful because more functionally monoclonal particles may result in higher quality signal, and thus improved SBS.

Another method of obtaining higher super-Poisson distributions is to have seeding occur quickly, followed by a delay among the seeded target polynucleotide. The delay, termed “kinetic delay” because it is thought to arise through the biochemical reaction kinetics, gives one seeded target polynucleotide an earlier start over the other seeded targets. Exclusion amplification works by using recombinase to facilitate the invasion of primers (e.g., primers attached to a substrate region) into double-stranded DNA (e.g., a target polynucleotide) when the recombinase mediates a sequence match. The present particles and methods may be adapted for use with recombinase to facilitate the invasion of the present amplification primers and orthogonal amplification primers into the present target polynucleotides when the recombinase mediates a sequence match. Indeed, the present compositions and methods may be adapted for use with any surface-based polynucleotide amplification methods such as thermal PCR, chemically denatured PCR, and enzymatically mediated methods (which may also be referred to as recombinase polymerase amplification (RPA), strand invasion, or ExAmp). For additional details and examples of amplification methods that are compatible with the present particles, see International Patent Application No. PCT/US2022/053005 to Ma et al., filed Dec. 15, 2022 and entitled “Hybrid Clustering,” and International Patent Application No. PCT/EP2023/058307 to Ma et al., filed Mar. 30, 2023 and entitled “Paired-End Resynthesis Using Blocked P5 Primers,” the entire contents of each of which are incorporated by reference herein.

From the foregoing, it will be appreciated that the present particles may be used to capture and amplify polynucleotides in solution, during sample preparation, before disposing the particles onto the surface of a flowcell. This has the potential to increase the quality of sequencing data (e.g., via improved clonality) and speed (e.g., because amplicons may be generated outside of the sequencing instrument). Additionally, once captured on the flowcell, the clustered particles may reduce any non-specific binding interaction within hydrogel-coated wells underneath the particles. In addition, the clustering in suspension may reduce strand invasion and pad-hopping, improving sequencing data quality. Additionally, the present particles may be used to recycle the flowcell after sequencing.

### WORKING EXAMPLES

The following examples are meant to be purely illustrative and not limiting of the present invention.

### Example 1

Magnetic particles between 250-300 nm in diameter and coated with DBCO were commercially obtained and processed in accordance with the example described with reference to FIGS. 1A-1D. The DBCO groups were reacted with the azide groups of PAZAM in a copper-free click reaction, which covalently bound the PAZAM onto the magnetic particles, forming a hydrogel coating. A mixture of P5 and P7 amplification primers was grafted to the PAZAM, using a copper-catalyzed click reaction between the azide groups of the PAZAM coating and the alkyne groups of the primers. After grafting, the magnetic particles were negatively charged (Zeta potential ˜30 mV).

The amplification primers then were used to generate a cluster of amplicons on the magnetic particle, in suspension, in accordance with the example described with reference to FIG. 2. More specifically, the PhiX library was used to generate clusters on the magnetic particles in the manner described with reference to FIG. 2, with the magnetic particles in suspension (“in-solution clustering,” or ISC). In-suspension seeding was performed for about 30 minutes, while the sample was maintained at 40° C. while being shaken at 1150 rpm in a thermomixer. The magnetic particles were disposed on a magnetic rack which separated the particles from one another, and washed. After 2-3 washing steps in a wash buffer, in-suspension clustering was carried out via strand invasion, as shown in FIG. 2. The reaction was conducted for 1 hour at 38° C., shaking at 1150 rpm.

Excess azide groups on a PAZAM-coated flowcell (FC) were converted into positively charged ammonium groups in a Staudinger reaction. The clustered magnetic particles were suspended in water and then deposited into nanowells within multiple lanes of the treated FC, where the positively charged surface helped to retain the particles in the nanowells via electrostatic interactions. After the nanowells were filled with clustered magnetic particles, the FC was sequenced using the HiSeq X sequencer from Illumina, Inc. A sufficient percentage of clusters were sufficiently monoclonal (% PF), had sufficient alignment, sufficient % Q30, and sufficient percent occupancy to perform sequencing.

These data show that the beads prepared according to this example were successfully integrated at the back end of the sample preparation through the seeding step. They have then been anchored to the surface of a flow cell and successfully sequenced. This demonstrate both compatibility with sample preparation and SBS workflow.

### Example 2

Magnetic particles were coupled to PAZAM and P5/P7 primers as described in Example 1. Excess azide groups on a PAZAM-coated flowcell (FC) were converted into positively charged ammonium groups in the manner described in Example 1. The magnetic particles, with primers coupled thereto, were suspended in water and then deposited into nanowells within multiple lanes of the treated FC, where the positively charged surface helped to retain the particles in the nanowells via electrostatic interactions. The PhiX library was used to generate clusters on the magnetic particles in the manner described with reference to FIG. 2, with the particles within the nanowells of the treated FC (“on-board clustering,” or OBC). After the nanowells were filled with clustered magnetic particles, the FC was sequenced using the HiSeq X sequencer from Illumina, Inc. A sufficient percentage of clusters were sufficiently monoclonal to perform sequencing (% PF).

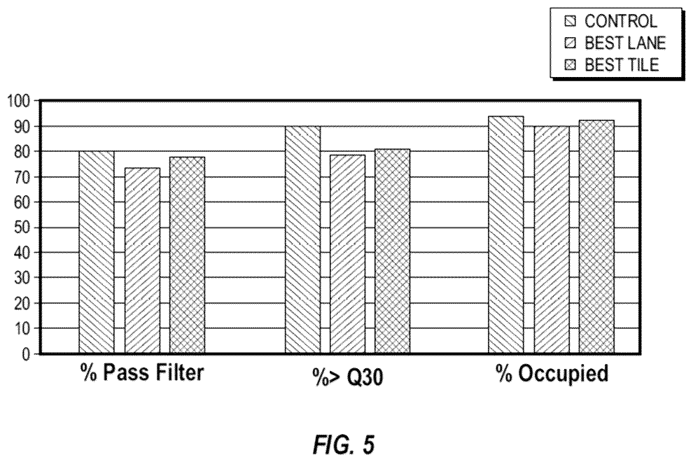

The clustered magnetic particles were shown to reproducibly withstand 300 cycles of sequencing and the paired end turn on the HiSeq X yielding similar sequencing metrics to that of a control lane which included grafted PAZAM, clustered with 300 pM library. FIG. 5 provides data showing sequencing metrics (pass filter (% PF), %>Q30, and occupancy percentages) for particles that were clustered on a flowcell in accordance with FIG. 2. From FIG. 5, it may be seen that the % PF, %>Q30, and occupancy percentages for the best lane and the best tile from lanes using OBC magnetic particles were similar to the control.

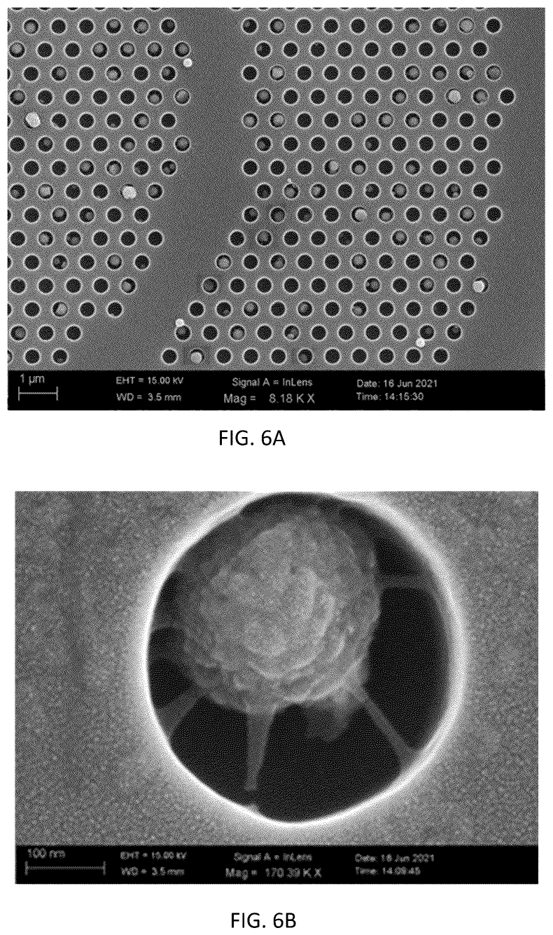

FIG. 6A is a scanning electron microscopy (SEM) image of 250 nm coated and grafted magnetic particles after a sequencing run, on a HiSeq X flowcell.

FIG. 6B is a SEM image of a 250 nm coated and grafted magnetic particle after sequencing run, on a HiSeq X flowcell. The magnetic particle was observed to be held securely in the well with tendrils, which was interpreted as meaning that interactions between the PAZAM on the magnetic particle and the PAZAM in the nanowell.

These data show that the beads prepared according to this example were successfully integrated at the back end of the sample preparation through the seeding step. They have then been anchored to the surface of a flow cell and successfully sequenced. This demonstrate both compatibility with sample preparation and SBS workflow.

### Example 3

The following workflow, consistent with FIG. 4, was performed.

Step 1. Library dsDNA was tagmented to form double-stranded fragments including cP5 and cP7 primers, and the dsDNA fragments were adsorbed onto commercially available SPRI beads.

Step 2. Particles which were coated in PAZAM and having P5 and P7 primers grafted thereto were added to and incubated with the larger SPRI beads to promote anchorage.

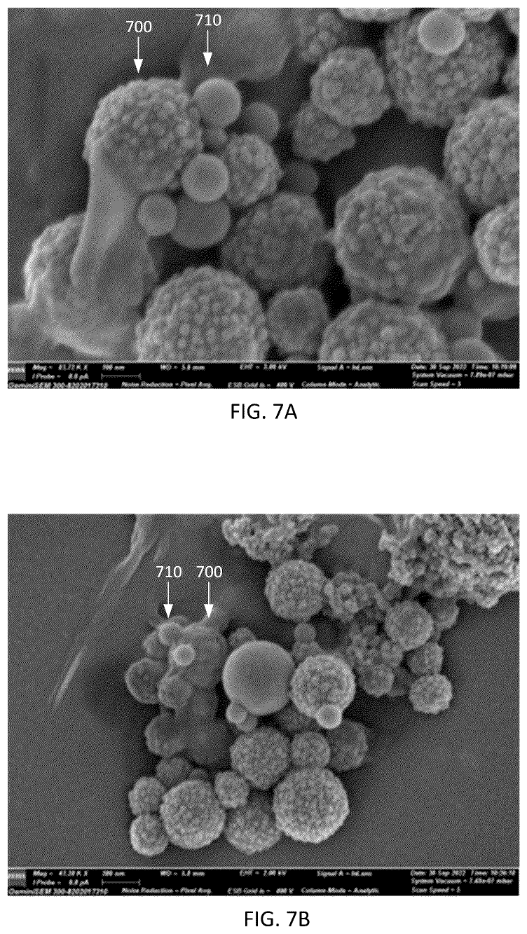

Step 3. Hybridization between the dsDNA fragments on the SPRI beads and the primers on the particles was performed via strand invasion. This step was catalysed by the addition of recombinases to the grafted articles during the anchorage step, to increase affinity and promote invasion. FIGS. 7A-7B are SEM images of SPRI beads 700 having PAZAM-coated and grafted particles 710 coupled thereto via hybridization using recombinase.

Step 4. The hybridization of the libraries with the primers on the particles results in the seeding of the particle itself. Due to the small contact area between the 2 beads (theoretically, 1 single point; practically, a little larger) it is anticipated that at most a few libraries will be seeded for each bead generating a pseudo-monoclonal seeding step.

Step 5. The particles were then released from the larger SPRI beads by first-extension in followed by dehybridization in NaOH.

Step 6. After the seeding steps were performed, the larger magnetic SPRI beads were removed from the solution through magnetic separation (see FIG. 4), leaving the seeded particles in solution.

Step 7. The seeded particles were captured onto a flowcell, clustered on-board and sequenced.

This protocol was used to generate seeded particles which were subsequently captured onto a HiSeq X flowcell, clustered on board (without library input) and sequenced.

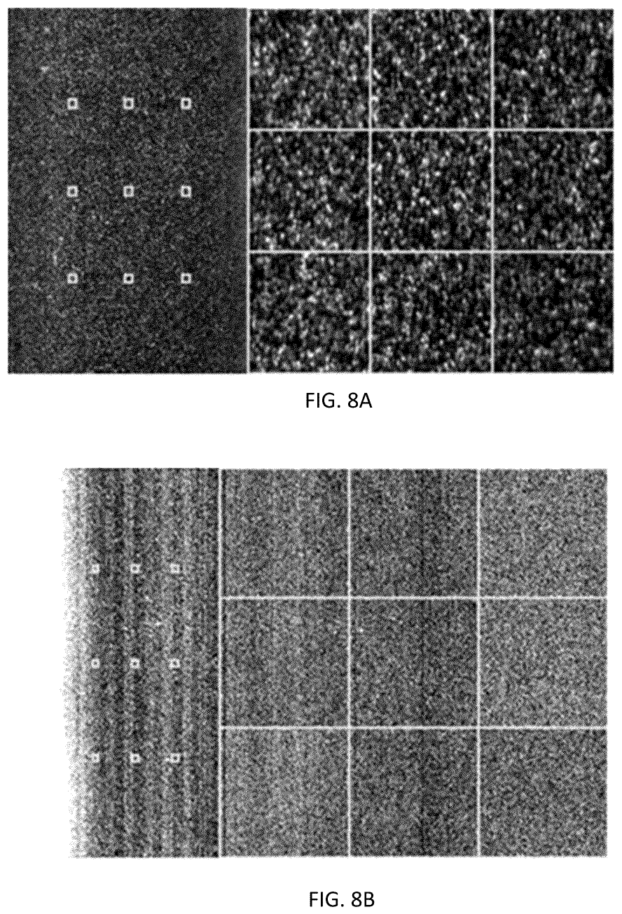

FIG. 8A is an image of a sequencing lane generated using the workflow described with reference to FIG. 4, followed by on-board clustering using a recombinase described with reference to FIG. 2. FIG. 8B is an image of a sequencing lane generated using the workflow described with reference to FIG. 8A, but without using recombinase, as a negative control. By comparing FIG. 8A to FIG. 8B, it may be understood that using the recombinase resulted in the generation of clusters which could be sequenced, whereas omitting the recombinase resulted in clusters not being formed.

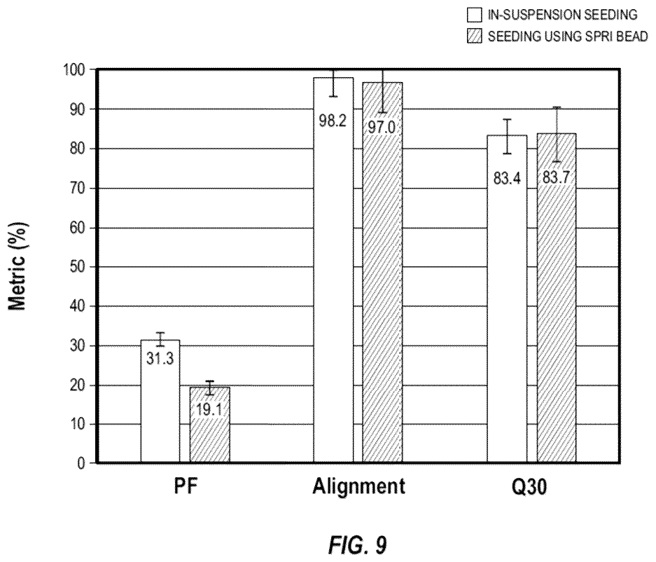

FIG. 9 provides data showing primary sequencing metrics between OBC particles which were seeded in suspension in accordance with FIG. 2 and Example 1, and OBC particles which were seeded in accordance with FIG. 4 and Example 3. From FIG. 9, it may be understood that either of these seeding modalities may be used to seed particles that then may be used to generate clusters that can be sequenced.

From the data of Examples 1, 2, and 3, it may be understood that particles which are seeded and amplified in a variety of manners provided herein, are compatible with sequencing by synthesis.

### ADDITIONAL COMMENTS

While various illustrative examples are described above, it will be apparent to one skilled in the art that various changes and modifications may be made therein without departing from the invention. The appended claims are intended to cover all such changes and modifications that fall within the true spirit and scope of the invention.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

1. A method of modifying a magnetic particle comprising a first functional group, the method comprising:
contacting the magnetic particle with a first molecule comprising a polymer coupled to second and third functional groups, wherein the first functional group reacts with the second functional group to form a bond via which the polymer is coupled to the magnetic particle; and
contacting the magnetic particle with a second molecule, the second molecule comprising an oligonucleotide coupled to a fourth functional group, wherein the third functional group reacts with the fourth functional group to form a bond via which the oligonucleotide is coupled to the magnetic particle.

2. The method of claim 1, wherein the oligonucleotide comprises an amplification primer.

3. The method of claim 1, wherein the second and third functional groups are of the same type as one another.

4. The method of claim 1, wherein the second and third functional groups are of different types than one another.

5. The method of claim 1, wherein the polymer comprises a hydrogel.

6. The method of claim 5, wherein the hydrogel comprises polyacrylamide.

7. The method of claim 1, wherein the magnetic particle has a diameter of about 100 nm to about 300 nm.

8. A method of modifying a magnetic particle coupled to a polymer comprising a first functional group, the method comprising:
contacting the magnetic particle with a molecule comprising an oligonucleotide coupled to a second functional group, wherein the second functional group reacts with the first functional group to form a bond via which the oligonucleotide is coupled to the magnetic particle.

9. A composition made by the method of claim 1.

10. A composition, comprising:
a magnetic particle;
a polymer coupled to the magnetic particle via a reaction product of a first functional group coupled to the magnetic particle and a second functional group coupled to the polymer; and
a plurality of oligonucleotides coupled to the polymer via reaction products of third functional groups coupled to the polymer and fourth functional groups coupled to the oligonucleotides.

11. A method of using the composition of claim 10, the method comprising:
hybridizing a template polynucleotide to a first oligonucleotide of the plurality; and
amplifying the hybridized template polynucleotide using additional oligonucleotides of the plurality to generate a plurality of amplicons coupled to the polymer via reaction products of the third and fourth functional groups.

12. The method of claim 11, further comprising, after the amplifying, disposing the magnetic particle within a flow cell.

13. The method of claim 12, wherein disposing the magnetic particle within the flow cell comprises electrostatically attracting the magnetic particle to a region of the flow cell.

14. The method of claim 11, further comprising, before the amplifying, disposing the magnetic particle within a flow cell.

15. A device comprising:
a flow cell; and
the composition of claim 10 located within the flow cell.

16. The device of claim 15, wherein the flow cell comprises a positively charged surface to which the composition is noncovalently bound through an electrostatic force.

17. A composition, comprising:
a magnetic particle; and
a molecule coupled to the magnetic particle, the molecule comprising:
a polymer coupled to the magnetic particle via a reaction product of first and second functional groups; and
a plurality of polynucleotide amplicons coupled to the polymer via reaction products of third and fourth functional groups.

18. A device comprising:
a flow cell; and
the composition of claim 17 located within the flow cell.

19. The device of claim 18, wherein the flow cell comprises a positively charged surface to which the composition is noncovalently bound through an electrostatic force.

20. A method of using the device of claim 19, the method comprising:
sequencing the plurality of polynucleotide amplicons within the flow cell.

21-35. (canceled)
